(19) United States
(12) Patent Application Publication
(10) Pub. No.: US 2011/0137412 A1
(43) Pub. Date: Jun. 9, 2011

(54) DELIVERY DEVICE FOR BREAST IMPLANT SURGERY

(76) Inventor: Christopher P. Nyte, Issaquah, WA (US)

(21) Appl. No.: 12/959,634

(22) Filed: Dec. 3, 2010

Related U.S. Application Data
(60) Provisional application No. 61/266,921, filed on Dec. 4, 2009.

(51) Int. Cl. A61M 3/02; A61F 2/12

(52) U.S. Cl. 623/8; 604/257

(57) ABSTRACT

An irrigation device is adapted to be placed into the body with and at the same time as a breast implant. The device delivers irrigation solution and/or antibiotic gels through small holes directly to the anterior (valve side) surface of a saline filled breast implant. The remaining implant surfaces, breast pocket and underside of the breast tissue will be likewise irrigated at the same time. By placing irrigation solutions and/or antibiotic gels directly onto the surface of the filled breast implant, bacterial biofilms will be targeted directly and eradicated, thereby reducing the incidence of capsule contracture and infection. The device is inserted with the breast implant, irrigates its surface and surround pocket when filled, and is removed with the breast implant fill tube at the end of the procedure.

### CROSS-REFERENCE TO RELATED APPLICATIONS

This application claims the benefit of priority of U.S. Provisional patent application No. 61/266,921, filed Dec. 4, 2009, which is herein incorporated by reference.

### BACKGROUND OF THE INVENTION

The present invention relates to surgical tools and, more particularly, to a silicone device for delivery of irrigation solutions and/or antibiotic or other gels to a breast implant and surgical pocket during breast augmentation surgery.

Subclinical bacterial colonization in the form of biofilms on the surface of breast implants and within the surgically created breast pocket is thought to be a major factor contributing to breast capsule contracture and implant infection. There are presently no devices that deliver irrigation solutions and/or antibiotic or other gels directly to the surface of a filled breast implant or surgically created pocket containing a filled breast implant. Bacterial biofilms remain on the implant and to some extent, within the surgically created pocket, increasing the likelihood of capsule contracture and infection.

Current irrigation devices are external and do not interact with or irrigate the surface of a filled breast implant or pocket with a filled breast implant. Current systems for delivering irrigation solutions during breast augmentation surgery are relegated to dispersing fluid only into the surgically created breast pocket. There is no way to irrigate the surface of a filled breast implant after placement into the body unless one introduces an external irrigation device and/or manipulates the implant externally, thereby introducing the possibility of additional bacterial contamination or rupture of the implant.

Also, certain approaches to placing the breast implant, such as the trans-axillary (TA) and trans-umbilical (TU) approaches, do not allow adequate access for placement of irrigation solutions and/or gels without introducing external devices. This method risks adding additional contamination to the pocket and/or rupture of the implant.

As can be seen, there is a need for an apparatus that can deliver irrigation solutions and/or antibiotic or other gels directly to the surface of a filled saline breast implant.

### SUMMARY OF THE INVENTION

In one aspect of the present invention, an irrigation device comprises an irrigation tube; a bladder connected to a first end of the irrigation tube, wherein the irrigation tube fluidly connects to an inside of the bladder; a connector attached to a second, opposite end of the irrigation tube; a through hole formed in a central region of the bladder; and a plurality of dispersal holes formed through the bladder.

In another aspect of the present invention, a method for performing a breast augmentation surgery comprises threading an implant fill tube through a through hole formed in a bladder, wherein an interior of the bladder is fluidly connected to an irrigation tube and a plurality of dispersal holes are formed through the bladder; attaching the implant fill tube to an inlet of a breast implant; resting the bladder on the implant to form a bladder/implant assembly; inserting the bladder/implant assembly into a patient; filling the breast implant; passing a solution or gel through the irrigation tube, into the bladder, through the dispersal holes and onto the breast implant; and removing the implant fill tube and the bladder from the patient simultaneously.

These and other features, aspects and advantages of the present invention will become better understood with reference to the following drawings, description and claims.

### BRIEF DESCRIPTION OF THE DRAWINGS

FIG. 1 is a front view of an irrigation device according to an exemplary embodiment of the present invention;

FIG. 2 is a perspective view of the irrigation device of FIG. 1, in use;

FIG. 3 is a top view of the irrigation device of FIG. 1, in use; and

FIG. 4 is a cross-sectional view taken along line 4-4 of FIG. 3.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides an irrigation device adapted to be placed into the body with and at the same time as a breast implant. The device delivers irrigation solution and/or antibiotic or other gels through small holes directly to the anterior (valve side) surface of a saline filled breast implant. The remaining implant surface, breast pocket and underside of the breast tissue will be likewise irrigated at the same time. By placing irrigation solutions and/or antibiotic gels directly onto the surface of the filled breast implant, bacterial biofilms will be targeted directly and eradicated, thereby reducing the incidence of capsule contracture and infection. The device is inserted with the breast implant, irrigates its surface and surround pocket when filled, and is removed with the breast implant fill tube at the end of the procedure.

The irrigation device, in its sterile form, is connected with the breast implant fill tube assembly and introduced into the body with the deflated breast implant at the same time. Irrigation solutions and/or gels can then be delivered through a silicone tube leading to a silicone bladder located on the central valve surface of a filled breast implant and held firmly in place by the breast implant fill tube. The irrigation solutions and/or antibiotic gels can then be dispersed onto the surface of the breast implant without additional manipulation of the implant and without introducing an external irrigation device.

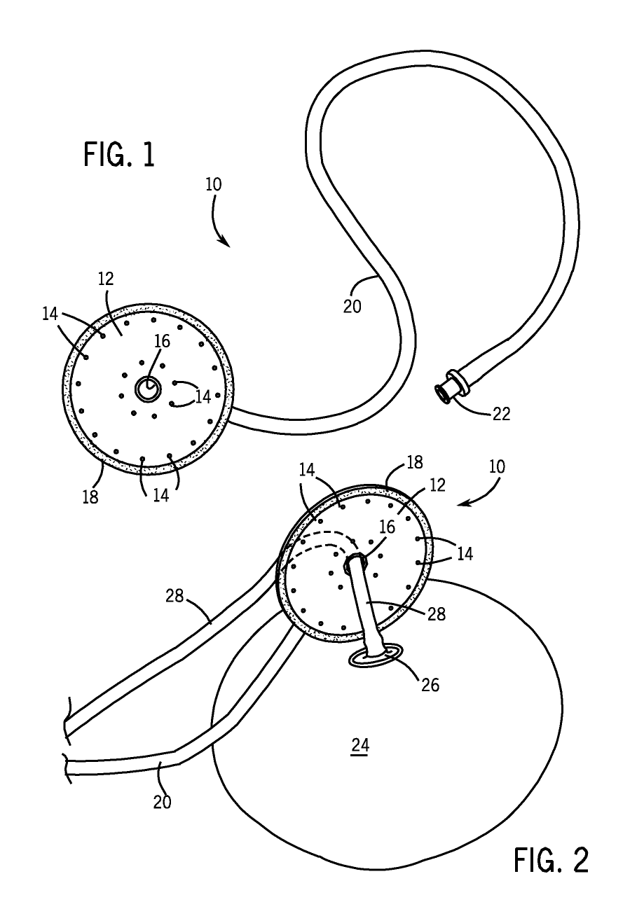

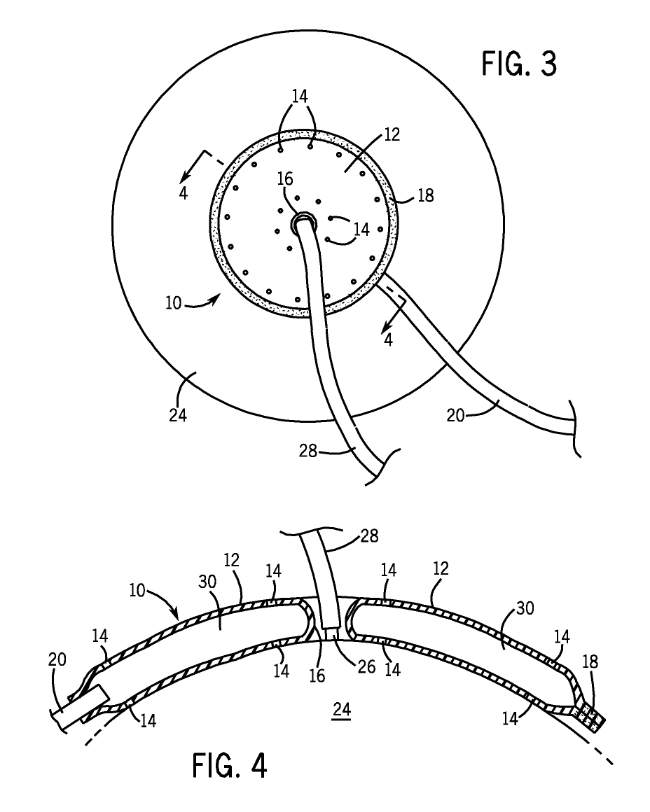

Referring to FIGS. 1 through 4, an irrigation device 10 may include an irrigation bladder 12, an irrigation tube 20 fluidly connected to an inside 30 of the irrigation bladder 12 at a first end thereof, and a connector 22 at a second, opposite end of the irrigation tube 20. In some embodiments, the irrigation tube 20 may attach to the irrigation bladder 12 at an outside edge 18 thereof. In other embodiments, the irrigation tube 20 may connect to the irrigation bladder 12 at any point on either side and at any distance from the outer edge of the irrigation bladder 12. The connector 22 may be, for example, a Luer-Lock hub. The connector 22 may be either a male or female oriented connector. The connector 22 may be made of, for example, medical grade plastic or metal.

The irrigation bladder 12 may have a first side and a second side sealed along the outside edge 18 thereof to form a pocket on the inside 30 of the irrigation bladder 12. Typically, the first and second sides are identical. The irrigation bladder 12 may be formed of a medical grade rubber or soft plastic, such as medical grade silicone. The thickness of each side of the irrigation bladder 12 may be from about 0.1 mm to about 0.75 mm, typically about 0.5 mm. The shape of the irrigation bladder 12 is typically circular, with a diameter from about 1 to 5 inches, typically about 2.5 inches. Other shapes, such as oval, elliptical, or the like, may be used for the irrigation bladder 12. The irrigation bladder 12 may include a central, circular through hole 16. The through hole 16 may have a diameter from about 5 to about 9 mm, typically about 7 mm.

The irrigation bladder 12 may include a plurality of solution dispersal holes 14 in each of its sides for dispersing irrigation solutions and/or antibiotic gels. These dispersal holes 14 may be arranged near the outer edge of the irrigation bladder 12 and near the through hole 16. Dispersal holes 14 may be located about 2 mm from the outer edge 18 on the surface of the irrigation bladder 12, and about 5 mm apart from each other. Dispersal holes 14 may also be located about 2 mm from the through hole 16 on the surface of the irrigation bladder, and about 5 mm apart from each other. Other patterns for the dispersal holes 14 may be utilized. In some embodiments, the dispersal holes 14 on one side of the irrigation bladder 12 may be offset from the dispersal holes 14 on the other side of the irrigation bladder 12. The dispersal holes 14 may have a diameter from about 0.5 to about 3 mm, typically about 1 mm.

A surgeon trained to perform breast augmentation would perform the surgery in his or her usual manner or approach. After the surgeon surgically creates a pocket to accept a saline breast implant and then irrigates the pocket using an external irrigation source, the implant is prepared in the usual manner by removing the sterile implant from its box. Prior to connecting an implant fill tube 28 to an implant valve 26, the surgeon can thread the implant fill tube 28 through the through hole 16 of the irrigation bladder 12 and then into an inlet 26 of a breast implant 24. The irrigation bladder 12 may be centered on and rest on the surface of the breast implant 24 in its unfilled form, prior to placement into the body. Next, the surgeon can align the implant fill tube 28 with the irrigation tube 20 so that they run parallel with each other. Next, air can be removed from the implant 24 in the usual fashion. The irrigation device 10/implant 24 assembly can be folded or rolled up in the usual fashion in preparation for insertion into the patient. The assembly can be placed into the patient using the same method as the implant alone would be placed. Once the assembly is in the surgically created pocket, the surgeon can fill the breast implant in the usual manner through the implant fill tube 28. Once filled, the surgeon can make any usual adjustments to the implant and implant pocket. Next, with the implant 24 filled, the surgeon can connect the connector 22 to a source of irrigation solution and/or antibiotic or other gels. The surgeon can then deliver the solutions and/or gels through the irrigation tube 20, into the irrigation bladder 12, through the dispersal holes 14 onto the surface of the breast implant 24. The undersurface of the breast tissue and the breast pocket would also receive irrigation solutions and/or antibiotic or other gels in the same fashion. The irrigation step can be repeated as desired. After irrigation, the surgeon can grasp and remove both the implant fill tube 28 and the irrigation tube 20 simultaneously from the surgical pocket. Hence, the implant tube 28 and the irrigation device 10 would be removed at the same time and in concert with each other. The surgeon would then conclude the surgery in his or her usual fashion.

It should be understood, of course, that the foregoing relates to exemplary embodiments of the invention and that modifications may be made without departing from the spirit and scope of the invention as set forth in the following claims.

What is claimed is:

1. An irrigation device comprising:
an irrigation tube;
a bladder connected to a first end of the irrigation tube, wherein the irrigation tube fluidly connects to an inside of the bladder;
a connector attached to a second, opposite end of the irrigation tube;
a through hole formed in a central region of the bladder; and
a plurality of dispersal holes formed through the bladder.

2. The irrigation device of claim 1, wherein the plurality of dispersal holes are formed in both a first side and a second side of the bladder.

3. The irrigation device of claim 2, wherein the plurality of dispersal holes include a first row of dispersal holes located about 2 mm from an outer edge of the bladder, and a second row of dispersal holes located about 2 mm from the through hole in the bladder.

4. The irrigation device of claim 1, wherein the bladder is a circular bladder having a diameter from about 1 to about 5 inches.

5. The irrigation device of claim 4, wherein the through hole is a circular through hole having a diameter from about 5 to 9 mm.

6. The irrigation device of claim 1, wherein the connector is a luer-type connector.

7. The irrigation device of claim 1, wherein the irrigation tube is attached to an outer edge of the bladder.

8. A method for performing a breast augmentation surgery, the method comprising:
threading an implant fill tube through a through hole formed in a bladder, wherein an interior of the bladder is fluidly connected to an irrigation tube and a plurality of dispersal holes are formed through the bladder;
attaching the implant fill tube to an inlet of a breast implant;
resting the bladder on the implant to form a bladder/implant assembly;
inserting the bladder/implant assembly into a patient;
filling the breast implant;
passing a solution or gel through the irrigation tube, into the bladder, through the dispersal holes and onto the breast implant; and
removing the implant fill tube and the bladder from the patient simultaneously.

9. The method of claim 8, further comprising attaching a connector to one end of the irrigation tube and supplying the solution or gel from a source through the connector.

10. The method of claim 8, further comprising repeating the step of passing the solution or gel onto the breast implant.
